(19) United States
(12) Patent Application Publication
(10) Pub. No.: US 2014/0342379 A1
(43) Pub. Date: Nov. 20, 2014

(54) Means and Methods for Assessing Gonadal Toxicity

(71) Applicant: BASF SE, Ludwigshafen (DE)

(72) Inventors: Tilman B. Walk, Kleinmachnow (DE); Bennard van Ravenzwaay, Altrip (DE); Werner Mellert, Hassloch (DE); Eric Fabian, Speyer (DE); Volker Strauss, Bad Durkheim (DE); Hennicke Kamp, Bischheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/344,488

(22) PCT Filed: Sep. 14, 2012
(86) PCT No.: PCT/IB2012/054799

§ 371 Date: Mar. 12, 2014

Related U.S. Application Data
(60) Provisional application No. 61/534,397, filed on Sep. 14, 2011.

(30) Foreign Application Priority Data
Sep. 14, 2011 (EP) 11181194.9

(51) Int. Cl. G01N 33/52

(52) U.S. Cl. CPC G01N 33/52; G01N 2500/04; G01N 2800/36; H01J 49/26
USPC 435/7.92; 435/287.2

(57) ABSTRACT

The present invention pertains to the field of diagnostics for gonadal toxicity and toxicological assessments for risk stratification of chemical compounds. Specifically, it relates to a method for diagnosing gonadal toxicity. It also relates to a method for determining whether a compound is capable of inducing such gonadal toxicity in a subject and to a method of identifying a drug for treating gonadal toxicity. Furthermore, the present invention relates to a device and a kit for diagnosing gonadal toxicity.

The present invention pertains to the field of diagnostics for gonadal toxicity and toxicological assessments for risk stratification of chemical compounds. Specifically, it relates to a method for diagnosing gonadal toxicity. It also relates to a method for determining whether a compound is capable of inducing such gonadal toxicity in a subject and to a method of identifying a drug for treating gonadal toxicity. Furthermore, the present invention relates to a device and a kit for diagnosing gonadal toxicity.

Gonadal toxicity encompasses an impairment, disease or disorder of the gonads, i.e. the testes or ovaries. Thus, gonadal toxicity includes based on the gender either ovary toxicity or testicular toxicity. The said gonadal toxicity may be a chemically induced, enhanced or driven gonadal toxicity.

The ovaries are paired organs and integrated part of the reproductive and of the endocrine system. The ovary is a complex organ with three major functions: production of fertile oocytes, synthesis of steroid hormones, and synthesis of regulatory proteins. These complex functions are reflected in the diversity of structures and their constituent cell types, the follicle with its oocyte, the corpus luteum and the interstitial gland. The primordial follicles are formed during embryonic development and represent a reserve. A species-dependent number of follicles ripen during each estrous cycle for ovulation. The morphology alters according to the different phases of estrous cycle, the age and altered endocrine functions. After ovulation, corpora lutea are formed, which are a transient endocrine gland but in the absence of pregnancy, it regresses. The ovaries also contain interstitial gland tissue with cells undergoing cyclic morphological changes and classified into several distinct morphological types of steroid producing cells and cells secreting androgens.

With regards to the functional diversity and the complexity of its hormonal regulation providing a number of potential sites for chemical disturbance, ovarian toxicity may become apparent in a subject by various disorders, diseases or medical conditions. The range of morphological alteration, which may be detected is diverse and reflects the morphological features. The injuries are located at the surface epithelium, the follicles, the corpora lutea and/or the interstitial glands. Many non-neoplastic ovarian lesions are characterized by an increase or decrease in different major tissue components, i.e. corpora lutea, die varying stages of developing follicles, and the interstitial gland. In contrast, neoplastic lesions are not always clearly differentiated due to die pluripotential nature of ovarian tissue and its ability to manifest as a wide diversity of cell types not normally found in die mature ovary, e.g. Sertoli cell tumors and germ cell tumors.

There is a wide range of non neoplastic lesions consisting of epithelial (mesothelial) and tubular hyperplasia, oocyte destruction, arrest of follicular development up to follicular atresia, follicular and interstitial atrophy, supraovulation, follicular luteinisation, follicular cysts, reduction/absence of corpora lutea, increased/persistence of corpora lutea, vacuolar degeneration of corpora lutea, sertoliform tubular hyperplasia, interstitial gland hypertrophy/hyperplasia atrophy, disturbance of estrous cycle and mesovarial smooth muscle hyperplasia.

Ovarian tumors can be subdivided into five broad categories, including epithelial tumors, sex cord-stromal tumors, germ cell tumors, tumors derived from nonspecialized soft tissues of the ovary, and tumors metastatic to the ovary from distant sites. The epithelial tumors of the ovary include cystadenomas and cystadenocarcinomas, tubulostromal adenomas, and mesothelioma. The tubular adenomas are the most important of the ovarian tumors in mice but they are uncommon in rats, rare in other animal species, and not recognized in the ovaries of women. Another important group of ovarian tumors are those derived from the sex cords and/or ovarian stroma. These include the granulosa cell tumors, luteoma, thecoma, Sertoli cell tumor, tubular adenoma with contributions from ovarian stroma, and undifferentiated sex cord-stromal tumors. The granulosa cell tumor is the most common of this group and may develop within certain tubular or tubulostromal adenomas following a long-term perturbation of endocrine function associated with genic deletion, irradiation, oocytotoxic chemicals, and neonatal thymectomy.

Due to the diversity of possible actions of agents to induce ovary toxicity, the assessment of disorders is a rather complex process. The current methods usually comprise clinical investigations, pathological and histopathological investigations as well as a biochemical and hormone analysis. Major drawbacks of the histopathological assessments are that they are invasive, and even when combined with the clinical pathology measurements or hormone analysis they are less reliable because they are in part based on the individual interpretations of toxicologist carrying out the investigations or the selected methods for hormone measurements.

The importance to investigate disorders of the ovary may become apparent if one considers that the ovary are integrated parts of the reproductive and of the endocrine system and as such can be impaired by chemically induced lesions. Moreover, chemical compounds which are used in any kind of industry in the European Community, e.g., will now need to comply with REACH (Registration, Evaluation and Authorisation of Chemicals). it will be understood that the potential of a chemical compound to induce disorders of the adrenal gland cortex will be deemed as a high risk for the compound and, consequently, the compound will be available only for limited applications and when obeying high security standards. (see Capen C C (2001) Overview of structural and functional lesions in endocrine organs of animals, Toxicol. Pathol., 29, 8-33; Capen C C (2008) Toxic responses of the endocrine system, Chapter 21, in: Casarett & Doull's Toxicology, The basic science of poisons, Klaassen C D (ed.), McGraw-Hill P, 7th revised edition, New York (2008); Capen C C, Martin S L (1989) Mechanisms that lead to disease of the endocrine system in animals, Toxicol. Pathol., 17, 234-249; Lewis D J, Gopinath C (1998) The female reproductive system, Chapter 13, 407-428, in: Target organ pathology, a basic text, Turton J and Hooson J (eds) Taylor & Francis, London, United Kingdom, 1998; Mattison D R, Thomford P J (1989) The mechanisms of action of reproductive toxicants, Toxicol. Pathol., 17, 364-376; Foster P M D, Gray L E (2008) Toxic responses of the reproductive system, Chapter 20, 761-806, in: Casarett & Doull's Toxicology, The basic science of poisons, Klaassen C D (ed.), McGraw-Hill P, 7th revised edition, New York (2008)

The testes are paired organs and integrated part of the reproductive and of the endocrine system. The testes consist of the tubular and the interstitial compartment as two morphological and functionally distinct areas. The seminiferous tubules are surrounded by interstitial tissue composed of connective tissue containing the testosterone producing Leydig cells, macrophages, lymph and blood vessels. The seminiferous tubules are lined by a stratified epithelium, which consists of germ cells at various stages of development and by Sertoli cells providing structural and nutritional support. The primary functions of the testes are the production of male germ cells, the sperms and male sex steroids hormones including testosterone and dihydrotestosterone but also small amounts of estrogens. Both functions are under the control of gonadotropic hormones produced by the pituitary.

Testicular spermatogenesis comprises a synchronized development of several generations of germ cells involving spermatogonial mitosis, spermatocyte meiosis, and morphological transformation of the undifferentiated spermatids into specialized motile sperm. Throughout this development, the germ cells are embedded within the cytoplasmic processes of the Sertoli cell. The Sertoli cell transfers essential molecules to the germ cells from the interstitial fluid, synthesizes essential substrates for germ cell metabolism, and generally regulates the progress of spermatogenesis through to completion. The release of the sperm is an active process that requires the separation of the specialized junctions between Sertoli cell and spermatid. The newly formed sperm are released into the seminiferous tubule fluid and transported via the rete testis into the efferent ducts and into the epididymis. Spermatogenesis relies on the coordinated support and interactions of the germ cells, Sertoli cells, Leydig cells, peritubular cells, interstitial macrophages, and the blood vasculature. Overall regulation of the process is mediated through the hypothalamic-pituitary-Leydig cell endocrine axis, but equally important is the local regulation of cellular function through paracrine factors.

Testicular toxicity may become apparent in a subject by various disorders, diseases or medical conditions as the functional diversity of the male reproductive system and the complexity of its hormonal regulation provide a number of potential sites for chemical disturbance. Injury may result in reduced sperm output or reduced testosterone secretion from the testis and interference with the transport of sperm through the duct system and the quality of the sperm available for fertilization may also be impaired. As a consequence of its role in spermatogenesis, one of the most common target cells for toxicity is the Sertoli cell. Any functional compromise of this cell will have rapid secondary effects on the survival of the dependent germ cells. Death of specific populations of germ cells may be seen in the case of cytotoxic agents that affect the mitotic divisions or the glycol ethers, which affect only pachytene and dividing spermatocytes. Leydig cell function is susceptible to any agent that interferes with the steroidogenic pathway or with circulating levels or receptor binding of its regulatory hormones, luteinizing hormone (LH), and prolactin.

Regardless of the primary site of toxic injury, most testicular toxicants will cause germ cell degeneration and depletion to a greater or lesser extent. One of the most common morphological responses of the Sertoli cell to injury is vacuolation with subsequent germ cell degeneration, disorganization, or exfoliation. Depending on the severity and duration of the Sertoli cell damage, the germ cell depletion may be partial or complete, but the Sertoli cells will remain intact as they are resistant to cell death. Many chemicals affecting spermatogenesis act indirectly through their effect on the Sertoli cell (e.g., DBCP, MEHP) rather than directly on the germ cells. Tetrahydrocannabinol (THC) acts at several sites in the reproductive system, including the Sertoli cells.

Fluid secretion in the testis serves an important function. Interstitial fluid composition is similar to that of testicular blood plasma but with the addition of high levels of testosterone and androstenedione secreted by the Leydig cells. Reduced fluid secretion and tubular contraction is seen with many testicular toxicants.

The major function of the interstitial Leydig cell is steroidogenesis and any toxicant that interferes with this pathway will produce functional disturbances in hormone balance. The interstitial Leydig cells frequently undergo proliferative changes (hyperplasia/neoplasia) with advancing age or following chronic exposure to large doses of chemicals. A number of nongenotoxic agents can produce Leydig cell hyperplasia (androgen receptor antagonists, reductase inhibitors, testosterone biosynthesis inhibitors, aromatase inhibitors, dopamine agonists, estrogen agonists/antagonists, and GnRH agonists). Nongenotoxic compounds that induce Leydig cell tumors in rats most likely have little relevance to humans under most exposure conditions because humans are quantitatively less sensitive than rats. Leydig (interstitial) cell tumors are among the more frequently occurring endocrine tumors in rodents in chronic toxicity/carcinogenicity studies. Rodent testicular tumors are classified into five general categories, including tumors derived from cells of the gonadal stroma, neoplasms of germ cell origin, tumors derived from adnexal structures or serous membranes, and, a group of tumors derived from the supporting connective tissues and vessels of the testis. Neoplasms of the gonadal stroma include benign and malignant tumors derived from Leydig (interstitial) cells, Sertoli cells of the seminiferous tubules, as well as a rare mixed tumor with an admixture of both cell types.

Due to the diversity of possible actions of agents to induce testicular toxicity, the assessment of disorders of the testes is a rather complex process. The current methods usually comprise clinical investigations, pathological and histopathological investigations as well as a biochemical and hormone analysis. However, the biomarkers are rather complex regulated and changes may sometimes occur even at rather progressed stages. Major drawbacks of the histopathological assessments are that they are invasive, and even when combined with the clinical pathology measurements or hormone analysis they are less reliable because they are in part based on the individual interpretations of toxicologist carrying out the investigations or the selected methods for hormone measurements.

The importance to investigate disorders of the testes may become apparent if one considers that the testes are integrated parts of the reproductive and of the endocrine system and as such can be impaired by chemically induced lesions. Moreover, chemical compounds which are used in any kind of industry in the European Community, e.g., will now need to comply with REACH (Registration, Evaluation and Authorisation of Chemicals). it will be understood that the potential of a chemical compound to induce disorders of the adrenal gland cortex will be deemed as a high risk for the compound and, consequently, the compound will be available only for limited applications and when obeying high security standards (see Capen C C (2001) Overview of structural and functional lesions in endocrine organs of animals, Toxicol. Pathol., 29, 8-33; Creasy D M (1998) The male reproductive system, Chapter 12, 371-405, in: Target organ pathology, a basic text, Turton J and Hooson J (eds) Taylor & Francis, London, United Kingdom, 1998; Creasy D M (2001) Pathogenesis of male reproductive toxicity, Toxicol. Pathol., 29, 64-76; Foley G L (2001) Overview of male reproductive pathology, Toxicol. Pathol., 29, 49-63; Foster P M D, Gray L E (2008) Toxic responses of the reproductive system, Chapter 20, 761-806, in: Casarett & Doull's Toxicology, The basic science of poisons, Klaassen C D (ed.), McGraw-Hill P, 7th revised edition, New York (2008); Heindel J J, Treinen K A (1989) Physiology of the male reproductive system: Endocrine, paracrine and autocrine regulation, Toxicol. Pathol., 17, 411-445; Mattison D R, Thomford P J (1989) The mechanisms of action of reproductive toxicants, Toxicol. Pathol., 17, 364-376; Utiger R D (2010) Anatomy of the testis in Encyclopedia Britannica. Retrieved Oct. 26, 2010, from Encyclopedia Britannica Online: http://www.britannica.com/EBchecked/topic/588769/testis).

Sensitive and specific methods for assessing the toxicological properties of a chemical compound and, in particular, gonadal toxicity, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing gonadal toxicity comprising:

(a) determining the amount of at least one biomarker selected from any one of Tables 1a, 1b, 3a, 3b, 4a, 4b, 5a, 5b, 6a or 6b in a test sample of a subject suspected to suffer from gonadal toxicity, and
(b) comparing the amounts determined in step (a) to a reference, whereby gonadal toxicity is to be diagnosed.

In a preferred embodiment of the aforementioned method said subject has been brought into contact with a compound suspected to be capable of inducing gonadal toxicity.

The present invention also relates to a method of determining whether a compound is capable of inducing gonadal toxicity in a subject comprising:

(a) determining in a sample of a subject which has been brought into contact with a compound suspected to be capable of inducing gonadal toxicity the amount of at least one biomarker selected from any one of Tables 1a, 1b, 3a, 3b, 4a, 4b, 5a, 5b, 6a or 6b; and
(b) comparing the amounts determined in step (a) to a reference, whereby the capability of the compound to induce gonadal toxicity is determined.

In a preferred embodiment of the aforementioned method said compound is at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

In another preferred embodiment of the methods of the present invention said reference is derived from (i) a subject or group of subjects which suffers from gonadal toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride. In a more preferred embodiment of said method essentially identical amounts for the biomarkers in the test sample and the reference are indicative for gonadal toxicity.

In another preferred embodiment of the methods of the present invention said reference is derived from (i) a subject or group of subjects known to not suffer from gonadal toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride. In a more preferred embodiment of said methods amounts for the biomarkers which differ in the test sample in comparison to the reference are indicative for gonadal toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for gonadal toxicity.

The present invention also contemplates a method of identifying a substance for treating gonadal toxicity comprising the steps of:

(a) determining in a sample of a subject suffering from gonadal toxicity which has been brought into contact with a candidate substance suspected to be capable of treating gonadal toxicity the amount of at least one biomarker selected from any one of Tables 1a, 1b, 3a, 3b, 4a, 4b, 5a, 5b, 6a or 6b; and
(b) comparing the amounts determined in step (a) to a reference, whereby a substance capable of treating gonadal toxicity is to be identified.

In a preferred embodiment of the aforementioned method said reference is derived from (i) a subject or group of subjects which suffers from gonadal toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride. In a more preferred embodiment of said method amounts for the biomarkers which differ in the test sample and the reference are indicative for a substance capable of treating gonadal toxicity.

In another preferred embodiment of the aforementioned method said reference is derived from (i) a subject or group of subjects known to not suffer from gonadal toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride. In a more preferred embodiment of the said methods essentially identical amounts for the biomarkers in the test sample and the reference are indicative for a substance capable of treating gonadal toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating gonadal toxicity.

The present invention also relates to the use of at least one biomarker selected from any one of Tables 1a, 1b, 3a, 3b, 4a, 4b, 5a, 5b, 6a or 6b or a detection agent for the said biomarker for diagnosing gonadal toxicity in a sample of a subject.

Moreover, the present invention relates to a device for diagnosing gonadal toxicity in a sample of a subject suspected to suffer therefrom comprising:

(a) an analyzing unit comprising a detection agent for at least one biomarker selected from any one of Tables 1a, 1b, 3a, 3b, 4a, 4b, 5a, 5b, 6a or 6b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby gonadal toxicity is diagnosed.

In a preferred embodiment of the device of the invention said stored reference is a reference derived from a subject or a group of subjects known to suffer from gonadal toxicity or a subject or group of subjects which has been brought into contact with at least one compound selected from the group consisting of 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an essentially identical amount of the at least one biomarker in the test sample in comparison to the reference is indicative for the presence of gonadal toxicity or wherein an amount of the at least one biomarker in the test sample which differs in comparison to the reference is indicative for the absence of gonadal toxicity.

In another preferred embodiment of the device of the invention said stored reference is a reference derived from a subject or a group of subjects known to not suffer from gonadal toxicity or a subject or group of subjects which has not been brought into contact with at least one compound selected from the group consisting of 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an amount of the at least one biomarker in the test sample which differs in comparison to the reference is indicative for the presence of gonadal toxicity or wherein an essential identical amount of the at least one biomarker in the test sample in comparison to the reference is indicative for the absence of gonadal toxicity.

Further, the present invention relates to a kit for diagnosing gonadal toxicity comprising a detection agent for the at least one biomarker and standards for the at least one biomarker the concentration of which is derived from a subject or a group of subjects known to suffer from gonadal toxicity or derived from a subject or a group of subjects known to not suffer from gonadal toxicity.

Encompassed by the invention is, in particular, a method for diagnosing ovary toxicity comprising:

(a) determining the amount of at least one biomarker selected from Table 6a or 6b in a test sample of a subject suspected to suffer from ovary toxicity, and
(b) comparing the amounts determined in step (a) to a reference, whereby ovary toxicity is to be diagnosed.

In a preferred embodiment of the aforementioned method said subject has been brought into contact with a compound suspected to be capable of inducing ovary toxicity.

The present invention also relates to a method of determining whether a compound is capable of inducing ovary toxicity in a subject comprising:

(a) determining in a sample of a subject which has been brought into contact with a compound suspected to be capable of inducing ovary toxicity the amount of at least one biomarker selected from Table 6a or 6b and
(b) comparing the amounts determined in step (a) to a reference, whereby the capability of the compound to induce ovary toxicity is determined.

In a preferred embodiment of the aforementioned method said compound is at least one compound selected from the group consisting of: Mifepristone and Raloxifene Hydrochloride.

In another preferred embodiment of the methods of the present invention said reference is derived from (i) a subject or group of subjects which suffers from ovary toxicity or (ii) a subject or group of subjects which has been brought into contact with at least one compound selected from the group consisting of: Mifepristone and Raloxifene Hydrochloride. In a more preferred embodiment of said method essentially identical amounts for the biomarkers in the test sample and the reference are indicative for ovary toxicity.

In another preferred embodiment of the methods of the present invention said reference is derived from (i) a subject or group of subjects known to not suffer from ovary toxicity or (ii) a subject or group of subjects which has not been brought into contact with at least one compound selected from the group consisting of: Mifepristone and Raloxifene Hydrochloride. In a more preferred embodiment of said methods amounts for the biomarkers which differ in the test sample in comparison to the reference are indicative for ovary toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for ovary toxicity.

The present invention also contemplates a method of identifying a substance for treating ovary toxicity comprising the steps of:

(a) determining in a sample of a subject suffering from ovary toxicity which has been brought into contact with a candidate substance suspected to be capable of treating ovary toxicity the amount of at least one biomarker selected from Table 6a or 6b; and
(b) comparing the amounts determined in step (a) to a reference, whereby a substance capable of treating ovary toxicity is to be identified.

In a preferred embodiment of the aforementioned method said reference is derived from (i) a subject or group of subjects which suffers from ovary toxicity or (ii) a subject or group of subjects which has been brought into contact with at least one compound selected from the group consisting of: Mifepristone and Raloxifene Hydrochloride In a more preferred embodiment of said method amounts for the biomarkers which differ in the test sample and the reference are indicative for a substance capable of treating ovary toxicity.

In another preferred embodiment of the aforementioned method said reference is derived from (i) a subject or group of subjects known to not suffer from ovary toxicity or (ii) a subject or group of subjects which has not been brought into contact with at least one compound selected from the group consisting of: Mifepristone and Raloxifene Hydrochloride. In a more preferred embodiment of the said methods essentially identical amounts for the biomarkers in the test sample and the reference are indicative for a substance capable of treating ovary toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating ovary toxicity.

The present invention also relates to the use of at least one biomarker selected from Tables 6a or 6b or a detection agent for the said biomarker for diagnosing ovary toxicity in a sample of a subject.

Moreover, the present invention relates to a device for diagnosing ovary toxicity in a sample of a subject suspected to suffer therefrom comprising:

(a) an analyzing unit comprising a detection agent for at least one biomarker selected from Table 6a or 6b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby ovary toxicity is diagnosed.

In a preferred embodiment of the device of the invention said stored reference is a reference derived from a subject or a group of subjects known to suffer from ovary toxicity or a subject or group of subjects which has been brought into contact with at least one compound selected from the group consisting of Mifepristone and Raloxifene Hydrochloride and said data processor executes instructions for comparing the amount of the at least one biomarker determined by the analyzing unit to the stored reference, wherein an essentially identical amount of the at least one biomarker in the test sample in comparison to the reference is indicative for the presence of ovary toxicity or wherein an amount of the at least one biomarker in the test sample which differs in comparison to the reference is indicative for the absence of ovary toxicity.

In another preferred embodiment of the device of the invention said stored reference is a reference derived from a subject or a group of subjects known to not suffer from ovary toxicity or a subject or group of subjects which has not been brought into contact with at least one compound selected from the group consisting of Mifepristone and Raloxifene Hydrochloride and said data processor executes instructions for comparing the amount of the at least one biomarker determined by the analyzing unit to the stored reference, wherein an amount of the at least one biomarker in the test sample which differs in comparison to the reference is indicative for the presence of ovary toxicity or wherein an essential identical amount of the at least one biomarker in the test sample in comparison to the reference is indicative for the absence of ovary toxicity.

Further, the present invention relates to a kit for diagnosing ovary toxicity comprising a detection agent for the at least one biomarker and standards for the at least one biomarker the concentration of which is derived from a subject or a group of subjects known to suffer from ovary toxicity or derived from a subject or a group of subjects known to not suffer from ovary toxicity.

Encompassed by the invention is, further, a method for diagnosing testicular toxicity comprising:

(a) determining the amount of at least one biomarker selected from any one of Tables 1a, 1b, 3a, 3b, 4a, 4b, 5a or 5b in a test sample of a subject suspected to suffer from testicular toxicity, and
(b) comparing the amounts determined in step (a) to a reference, whereby testicular toxicity is to be diagnosed.

In a preferred embodiment of the aforementioned method said subject has been brought into contact with a compound suspected to be capable of inducing testicular toxicity.

The present invention also relates to a method of determining whether a compound is capable of inducing testicular toxicity in a subject comprising:

(a) determining in a sample of a subject which has been brought into contact with a compound suspected to be capable of inducing testicular toxicity the amount of at least one biomarker selected from any one of Tables 1a, 1b, 3a, 3b, 4a, 4b, 5a or 5b in a test sample of a subject which has been brought into contact with a compound suspected to be capable of inducing testicular toxicity; and
(b) comparing the amounts determined in step (a) to a reference, whereby the capability of the compound to induce testicular toxicity is determined.

In a preferred embodiment of the aforementioned method said compound is at least one compound selected from the group consisting of: 17-alpha-Ethynylestradiol, Lysmeral, 2-Methoxyethanol, 2-Butoxyethanol, 2-Methylimidazole, Phenylbutazone and 2,5-Hexanedione.

In another preferred embodiment of the methods of the present invention said reference is derived from (i) a subject or group of subjects which suffers from testicular toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone and 2,5-Hexanedione. In a more preferred embodiment of said method essentially identical amounts for the biomarkers in the test sample and the reference are indicative for testicular toxicity.

In another preferred embodiment of the methods of the present invention said reference is derived from (i) a subject or group of subjects known to not suffer from testicular toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone and 2,5-Hexanedione. In a more preferred embodiment of said methods amounts for the biomarkers which differ in the test sample in comparison to the reference are indicative for testicular toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for testicular toxicity.

The present invention also contemplates a method of identifying a substance for treating testicular toxicity comprising the steps of:

(a) determining in a sample of a subject suffering from testicular toxicity which has been brought into contact with a candidate substance suspected to be capable of treating testicular toxicity the amount of at least one biomarker selected from any one of Tables 1a, 1b, 3a, 3b, 4a, 4b, 5a or 5b; and
(b) comparing the amounts determined in step (a) to a reference, whereby a substance capable of treating testicular toxicity is to be identified.

In a preferred embodiment of the aforementioned method said reference is derived from (i) a subject or group of subjects which suffers from testicular toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone and 2,5-Hexanedione. In a more preferred embodiment of said method amounts for the biomarkers which differ in the test sample and the reference are indicative for a substance capable of treating testicular toxicity.

In another preferred embodiment of the aforementioned method said reference is derived from (i) a subject or group of subjects known to not suffer from testicular toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone and 2,5-Hexanedione. In a more preferred embodiment of the said methods essentially identical amounts for the biomarkers in the test sample and the reference are indicative for a substance capable of treating testicular toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating testicular toxicity.

The present invention also relates to the use of at least one biomarker selected from any one of Tables 1a, 1b, 3a, 3b, 4a, 4b, 5a or 5b or a detection agent for the said biomarker for diagnosing testicular toxicity in a sample of a subject.

Moreover, the present invention relates to a device for diagnosing testicular toxicity in a sample of a subject suspected to suffer therefrom comprising:

(a) an analyzing unit comprising a detection agent for at least one biomarker selected from any one of Tables 1a, 1b, 3a, 3b, 4a, 4b, 5a or 5b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby testikular toxicity is diagnosed.

In a preferred embodiment of the device of the invention said stored reference is a reference derived from a subject or a group of subjects known to suffer from testicular toxicity or a subject or group of subjects which has been brought into contact with at least one compound selected from the group consisting of 17-alpha-Ethynylestradiol, Lysmeral, 2-Methoxyethanol, 2-Butoxyethanol, 2-Methylimidazole, Phenylbutazone, and 2,5-Hexanedione, said data processor executes instructions for comparing the amount of the at least one biomarker determined by the analyzing unit to the stored reference, wherein an essentially identical amount of the at least one biomarker in the test sample in comparison to the reference is indicative for the presence of testicular toxicity or wherein an amount of the at least one biomarker in the test sample which differs in comparison to the reference is indicative for the absence of testicular toxicity.

In another preferred embodiment of the device of the invention said stored reference is a reference derived from a subject or a group of subjects known to not suffer from testicular toxicity or a subject or group of subjects which has not been brought into contact with at least one compound selected from the group consisting of 17-alpha-Ethynylestradiol, Lysmeral, 2-Methoxyethanol, 2-Butoxyethanol, 2-Methylimidazole, Phenylbutazone, and 2,5-Hexanedione, and said data processor executes instructions for comparing the amount of the at least one biomarker determined by the analyzing unit to the stored reference, wherein an amount of the at least one biomarker in the test sample which differs in comparison to the reference is indicative for the presence of testicular toxicity or wherein an essential identical amount of the at least one biomarker in the test sample in comparison to the reference is indicative for the absence of testicular toxicity.

Further, the present invention relates to a kit for diagnosing testicular toxicity comprising a detection agent for the at least one biomarker and standards for the at least one biomarker the concentration of which is derived from a subject or a group of subjects known to suffer from testicular toxicity or derived from a subject or a group of subjects known to not suffer from testicular toxicity.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pre-treatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex vivo on a sample of a subject, i.e. not practised on the human or animal body.

The term “diagnosing” as used herein refers to assessing the probability according to which a subject is suffering from a condition, such as a intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Confirmation relates to the strengthening or substantiating a diagnosis of the condition or a predisposition for the condition already determined using other indicators or markers. Classification relates to (i) allocating the condition into different classes, e.g., corresponding to the strength of the symptoms accompanying the condition, or (ii) differentiating between different stages, disease or disorders accompanying the condition. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified.

The term “gonadal toxicity” as used herein relates to any damage or impairment of the gonads, i.e. the ovaries or testes, which results in an impaired gonadal function. Preferably, affected by gonadal toxicity are the reproduction related functions of the gonads. Preferably, gonadal toxicity as used herein is induced by or is the result of the administration of a chemical compound or drug, i.e. a so-called toxin-induced gonadal toxicity. More preferably, gonadal toxicity is ovary toxicity or testicular toxicity. Preferably, gonadal toxicity is accompanied by cell death and/or impairment of cellular functions in the gonads.

Ovary toxicity is, preferably, manifested by one or more diseases or disorders from the following group: Non-neoplastic lesions consisting of epithelial (mesothelial) and tubular hyperplasia, oocyte destruction, arrest of follicular development up to follicular atresia, follicular and interstitial atrophy, supraovulation, follicular luteinisation, follicular cysts, reduction/absence of corpora lutea, increased/persistence of corpora lutea, vacuolar degeneration of corpora lutea, sertoliform tubular hyperplasia, interstitial gland hypertrophy/hyperplasia atrophy, disturbance of estrous cycle, mesovarial smooth muscle hyperplasia or ovarian tumors. Ovarian tumors can be, preferably, subdivided into five broad categories, including epithelial tumors, sex cord—stromal tumors, germ cell tumors, tumors derived from nonspecialized soft tissues of the ovary, and tumors metastatic to the ovary from distant sites. The epithelial tumors of the ovary include cystadenomas and cystadenocarcinomas, tubulostromal adenomas, and mesothelioma. The tubular adenomas are the most important of the ovarian tumors in mice but they are uncommon in rats, rare in other animal species, and not recognized in the ovaries of women. Another important group of ovarian tumors are those derived from the sex cords and/or ovarian stroma. These include the granulosa cell tumors, luteoma, thecoma, Sertoli cell tumor, tubular adenoma with contributions from ovarian stroma, and undifferentiated sex cord—stromal tumors. The granulosa cell tumor is the most common of this group and may develop within certain tubular or tubulostromal adenomas following a long-term perturbation of endocrine function associated with genic deletion, irradiation, oocytotoxic chemicals, and neonatal thymectomy.

Ovary toxicity as used herein, preferably, refers to endocrine ovary toxicity. Such a toxicity may be accompanied by impaired estrogen and/or progesterone production and associated disorders.

Testicular toxicity is, preferably, manifested by one or more diseases or disorders from the following group: hyperplasia or neoplasia of interstitial Leydig cells or Leydig cell tumors. Leydig (interstitial) cell tumors are among the more frequently occurring endocrine tumors in rodents in chronic toxicity/carcinogenicity studies. Rodent testicular tumors are classified into five general categories, including tumors derived from cells of the gonadal stroma, neoplasms of germ cell origin, tumors derived from adnexal adnexal structures or serous membranes, and, a group of tumors derived from the supporting connective tissues and vessels of the testis. Neoplasms of the gonadal stroma include benign and malignant tumors derived from Leydig (interstitial) cells, Sertoli cells of the seminiferous tubules, as well as a rare mixed tumor with an admixture of both cell types.

Testicular toxicity as used herein, preferably, refers to testicular toxicity, Lysmeral-based toxicity, testes toxicity and/or testis degeneration.

Testicular toxicity is preferably diagnosed if a biomarker selected from Table 1a or 1 b is determined.

Lysmeral-based toxicity is preferably diagnosed if a biomarker selected from Table 3a or 3b is determined.

Testes toxicity is preferably diagnosed if a biomarker selected from Table 4a or 4b is determined.

Testis degeneration is preferably diagnosed if a biomarker selected from Table 5a or 5b is determined.

The symptoms and clinical signs of the aforementioned manifestations of gonadal toxicity are well known to the person skilled in the art and are described in detail in standard books of toxicology, e.g., H. Marquardt, S. G. Schäfer, R. O. McClellan, F. Welsch (eds.), “Toxicology”, Chapter 13: The Liver, 1999, Academic Press, London.

It was found in accordance with the present invention that a combination of more than one of the biomarkers listed in the Tables further strengthen the diagnosis since each of the biomarkers is an apparently statistically independent predictor for the diagnosis. Moreover, the specificity for gonadal toxicity is also significantly increased since influences from other tissues on the marker abundance are counterbalanced. Thus, the term “at least one” as used herein, preferably, refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

Particular preferred biomarkers to be determined either as single biomarkers or in the combinations recited below are listed as follows:

A preferred group or combination of biomarkers for diagnosing ovary toxicity comprises, essentially consists of or is selected from the following biomarkers: Cholesterol, Docosapentaenoic acid (C22:cis[7,10,13,16,19]5), Arachidonic acid (C20:cis[5,8,11,14]4), myo-Inositol, lipid fraction or Docosahexaenoic acid (C22:cis[4,7,10,13,16,19]6).

A preferred group or combination of biomarkers for diagnosing lysmeral-based toxicity comprises, essentially consists of or is selected from the following biomarkers: Lignoceric acid (C24:0), Ceramide (d18:1,C24:0), 3-O-Methylsphingosine (d18:1), Lysine or Sphingomyelin (d18:1,C24:0)

A preferred group or combination of biomarkers for diagnosing testes toxicity comprises, essentially consists of or is selected from the following biomarkers: Lignoceric acid (C24:0), Ceramide (d18:1,C24:0), Sphingomyelin (d18:1,C24:0), erythro-Sphingosine or Ceramide (d18:1,C24:1).

A preferred group or combination of biomarkers for diagnosing testicular toxicity comprises, essentially consists of or is selected from the following biomarkers: Lignoceric acid (C24:0), Cholesterol, Stearic acid (C18:0), Lysine or 3-O-Methylsphingosine (d18:1).

A preferred group or combination of biomarkers for diagnosing testis degeneration comprises, essentially consists of or is selected from the following biomarkers: Uracil, Glucose, myo-Inositol, Malate or Oleic acid (C18:cis[9]1).

Thus, preferably the at least one biomarker is, preferably, a biomarker selected from the aforementioned groups for the respective diseases or the at least one biomarker is a combination of biomarkers consisting or comprising the aforementioned biomarkers for a respective disease together. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, gonadal toxicity as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomeres of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties.

The term “test sample” as used herein refers to samples to be used for the diagnosis of gonadal toxicity by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing gonadal toxicity. A subject which has been brought into contact with a compound suspected to induce gonadal toxicity may, e.g., be a laboratory animal such as a rat which is used in a screening assay for, e.g., toxicity of compounds. A subject suspected to have been in contact with a compound capable of inducing gonadal toxicity may be also a subject to be diagnosed for selecting a suitable therapy. Preferably, a compound capable of inducing gonadal toxicity as used herein is 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)2 fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic con-version of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to gonadal toxicity.

Such references are, preferably, obtained from a sample derived from a subject or group of subjects which suffer from gonadal toxicity or from a sample derived from a subject or group of subjects which have/has been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride. A subject or group of subjects may be brought into contact with the said compounds by each topic or systemic administration mode as long as the compounds become bioavailable. Preferred modes of administration for the aforementioned compounds are described in the accompanying Examples, below.

Alternatively, but nevertheless also preferred, the reference may be obtained from sample derived from a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or a healthy subject or group of such subjects with respect to gonadal toxicity and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for gonadal toxicity because suitable reference results can be identified in the database once it has been confirmed (in the future) that the subject from which the corresponding reference sample was obtained (indeed) developed gonadal toxicity.

The term “comparing” refers to assessing whether the amount of the qualitative or quantitative determination of the at least one biomarker is identical to a reference or differs therefrom.

In case the reference results are obtained from a sample derived from a subject or group of subjects suffering from gonadal toxicity or a subject or group of subjects which has been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride, gonadal toxicity can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference, more preferably, the 50^{th}, 60^{th}, 70^{th}, 80^{th}, 90^{th} or 95^{th} percentile of the reference. A reference obtained from a sample derived from a subject or group of subjects suffering from gonadal toxicity or a subject or group of subjects which has been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride, can be applied in the methods of the present invention in order to diagnose gonadal toxicity or for determining whether a compound is capable of inducing gonadal toxicity in a subject. In such a case, preferably, an amount of the at least one biomarker which is essentially identical to the reference will be indicative for the presence of gonadal toxicity or a compound which is capable of inducing gonadal toxicity, while an amount of the at least one biomarker which differs from the reference will be indicative for the absence of gonadal toxicity or a compound which is not capable of inducing gonadal toxicity. Moreover, a reference obtained from a sample derived from a subject or group of subjects suffering from gonadal toxicity or a subject or group of subjects which has been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride, can be applied for identifying a substance for treating gonadal toxicity. In such a case, preferably, an amount of the at least one biomarker which differs from the reference will be indicative for a substance suitable for treating gonadal toxicity, while an amount of the at least one biomarker which is essentially identical to the reference will be indicative for a substance which is not capable of treating gonadal toxicity.

In case the reference results are obtained from a sample of a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or which does not suffer from gonadal toxicity, said gonadal toxicity can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference.

A reference obtained from a sample derived from a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or which does not suffer from gonadal toxicity can be applied in the methods of the present invention in order to diagnose the gonadal toxicity or for determining whether a compound is capable of inducing gonadal toxicity in a subject. In such a case, preferably, an amount of the at least one biomarker which differs from the reference will be indicative for the presence of gonadal toxicity or a compound which is capable of inducing gonadal toxicity, while an amount of the at least one biomarker which is essentially identical to the reference will be indicative for the absence of gonadal toxicity or a compound which is not capable of inducing gonadal toxicity. Moreover, a reference obtained from a sample derived from a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or which does not suffer from gonadal toxicity can be applied for identifying a substance for treating gonadal toxicity. In such a case, preferably, an amount of the at least one biomarker which is essentially identical to the reference will be indicative for a substance suitable for treating gonadal toxicity, while an amount of the at least one biomarker which differs from the reference will be indicative for a substance which is not suitable for treating gonadal toxicity.

Preferred references are those referred to in the accompanying Tables or those which can be generated following the accompanying Examples. Moreover, relative differences, i.e. increases (up-regulations) or decreases (down-regulations) in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Table 1a, 3a, 4a, 5a or 6a is increased with respect to a reference obtained from a sample derived from a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or which does not suffer from gonadal toxicity and, more preferably, a reference as indicated in the said Tables.

Preferably, the at least one biomarker when selected from Table 1b, 3b, 4b, 5b, or 6b is decreased with respect to a reference obtained from a sample derived from a subject or group of subjects which has not been brought into contact with 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or which does not suffer from gonadal toxicity and, more preferably, a reference as indicated in the said Tables.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating gonadal toxicity” refers to compounds which may directly interfere with the biological mechanisms inducing gonadal toxicity referred to elsewhere in this specification Alternatively, but also preferred the compounds may interfere with the development or progression of symptoms associated with the gonadal toxicity. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs.

It is to be understood that if the methods of the present invention are to be used for identifying drugs for the therapy of gonadal toxicity or for toxicological assessments of compounds (i.e. determining whether a compound is capable of inducing gonadal toxicity), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for gonadal toxicity (e.g. a query search). Thus, if an identical or similar data set can be identified in the data collection, the test data set will be associated with gonadal toxicity. The evaluation unit may also preferably comprise or be operatively linked to a further database with recommendations for therapeutic or preventive interventions or life style adaptations based on the established diagnosis of gonadal toxicity. Said further database can be, preferably, automatically searched with the diagnostic result obtained by the evaluation unit in order to identify suitable recommendations for the subject from which the test sample has been obtained in order to treat or prevent gonadal toxicity.

In a preferred embodiment of the device of the present invention, said stored reference is a reference derived from a subject or a group of subjects known to suffer from gonadal toxicity or a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an essentially identical amount of the at least one biomarker in the test sample in comparison to the reference is indicative for the presence of gonadal toxicity or wherein an amount of the at least one biomarker in the test sample which differs in comparison to the reference is indicative for the absence of gonadal toxicity.

In another preferred embodiment of the device of the present invention, said stored reference is a reference derived from a subject or a group of subjects known not to suffer from gonadal toxicity or a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an amount of the at least one biomarker in the test sample which differs in comparison to the reference is indicative for the presence of gonadal toxicity or wherein an essentially identical amount of the at least one biomarker in the test sample in comparison to the reference is indicative for the absence of gonadal toxicity.

The device, thus, can also be used without special medical knowledge by medicinal staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of at least one biomarker selected from any one of Tables 1a, 1b, 3a, 3b, 4a, 4b, 5a, 5b, 6a or 6b when present in solution or dissolved in a predefined volume of a solution resembles the amount of the at least one biomarker which is present (i) in a subject or a group of subjects known to suffer from gonadal toxicity or a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or (ii) derived from a subject or a group of subjects known to not suffer from therefrom or a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

Advantageously, it has been found in the study underlying the present invention that the amount of at least one biomarker as specified herein allows for diagnosing gonadal toxicity, specifically gonadal toxicity induced by 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride The specificity and accuracy of the method will be even more improved by determining an increasing number or even all of the aforementioned biomarkers. A change in the quantitative and/or qualitative composition of the metabolome with respect to these specific biomarkers is indicative for gonadal toxicity even before other signs of the said toxicity are clinically apparent. The morphological, physiological as well as biochemical parameters which are currently used for diagnosing gonadal toxicity are less specific and less sensitive in comparison to the biomarker determination provided by the present invention. Thanks to the present invention, gonadal toxicity of a compound can be more efficiently and reliably assessed. Moreover, based on the aforementioned findings, screening assays for drugs which are useful for the therapy of gonadal toxicity are feasible. In general, the present invention contemplates the use of at least one biomarker in a sample of a subject selected from the Tables 1a, 1b, 3a, 3b, 4a, 4b, 5a, 5b, 6a or 6b or a detection agent for said biomarker for diagnosing gonadal toxicity, for determining whether a compound is capable of inducing gonadal toxicity or for identifying a substance capable of treating gonadal toxicity. Further, the present invention, in general, contemplates the use of the at least one biomarker in a sample of a subject or a detection agent therefor for identifying a subject being susceptible for a treatment of gonadal toxicity. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1b, 3a, 3b, 4a, 4b, 5a, 5b, 6a or 6b. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for gonadal toxicity (e.g. a query search). Thus, if an identical or similar data set can be identified in the data collection, the test data set will be associated with gonadal toxicity. Consequently, the information obtained from the data collection can be used to diagnose gonadal toxicity based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising

(a) means for comparing characteristic values of at least one biomarker selected from the biomarkers listed in any one of Tables 1a, 1b, 3a, 3b, 4a, 4b, 5a, 5b, 6a or 6b comprised in a sample operatively linked to
(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for gonadal toxicity. Thus, the system of the present invention allows identifying whether a test data set is comprised by the data collection stored in the data storage medium. Consequently, the system of the present invention may be applied as a diagnostic means in diagnosing gonadal toxicity. In a preferred embodiment of the system, means for determining characteristic values of biomakers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

The following Examples are merely for the purposes of illustrating the present invention. They shall not be construed, whatsoever, to limit the scope of the invention in any respect.

### EXAMPLES

### Example

### Biomarkers Associated with Gonadal Toxicity

A group of each 5 male and female rats was dosed once daily with the indicated compounds (see Table 10, below for compounds, applied doses and administration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substances (compounds) in the Tables 1 to 9 below were dosed and administered as described in the Table 10 above.

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N2 atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatment duration and metabolite was determined by comparing means of the treated groups to the means of the respective untreated control groups using WELCH-test and quantified with treatment ratios versus control and p-values.

The identification of the most important biomarkers per toxicity pattern was done by a ranking of the analytes in the tables below. Therefore the metabolic changes in reference treatments of a given pattern (shown in the table) were compared with changes of the same metabolite in other unrelated treatments. For each metabolite T-values were obtained for the reference and control treatment and compared by the Welch test to asses whether these two groups are significantly different. The maximum absolute value of the respective TVALUE was taken to indicate the most important metabolite for the pattern.

The changes of the group of plasma metabolites being indicative for gonodal toxicity after treatment of the rats are shown in the following tables:

TABLE 1

| | 17-alpha- | | | | | | |
|---|---|---|---|---|---|---|---|
| | Ethynyl- | | | | | 2-Methoxy- | |
| | estradiol | | Lysmeral | | | ethanol | |
| Metabolite | m7 | m14 | m7 | m14 | m28 | m7 | m14 |

a: Markers for gonadal toxicity (testicular toxicity) in male rats;
Significant up-regulation changes (p-Value ≦0.1) are marked (*).
For some metabolites (marked with #), additional
information are provide in table 2.

| | | | | | | | |
|---|---|---|---|---|---|---|---|
| Lysine | 1.47 | 1.4* | 6.14* | 6.55* | 8.13* | 1.22* | 1.86* |
| Threonine | 1.44* | 1.85* | 1.46* | 1.74* | 2.15* | 1.72* | 2.93* |
| Histidine | 2.33* | 2.16* | 1.21* | 1.37* | 1.56* | 1.13 | 1.4* |

b: Markers for gonadal toxicity (testicular toxicity) in male rats;
Significant down-regulation changes (p-Value ≦0.1) are marked (*).
For some metabolites (marked with #), additional
information are provide in table 2.

| | | | | | | | |
|---|---|---|---|---|---|---|---|
| Lignoceric acid | NA | 0.47* | 0.36 | 0.52* | 0.6* | 0.61* | 0.46* |
| (C24:0) | | | | | | | |
| Cholesterol | 0.05 | 0.57* | 0.3 | 0.47* | 0.49* | 0.58* | 0.61* |
| Stearic acid | 0.18 | 0.67* | 0.34* | 0.51* | 0.55* | 0.8* | 0.84* |
| (C18:0) | | | | | | | |
| 3-O- | 0.06 | 0.35* | 0.22 | 0.45* | 0.48* | 0.53* | 0.67* |
| Methylsphingosine | | | | | | | |
| (d18:1)# | | | | | | | |
| erythro- | 0.03 | 0.45* | 0.21 | 0.51* | 0.51* | 0.57* | 0.67* |
| Sphingosine | | | | | | | |
| 3-Hydroxybutyrate | 0.71 | 0.23* | 0.24* | 0.29* | 0.52* | 0.45* | 0.24* |
| Coenzyme Q10 | 0.09* | 0.36* | 0.33* | 0.24* | 0.37* | 0.7* | 0.49* |
| Coenzyme Q9 | 0.09* | 0.86 | 0.21* | 0.27* | 0.31* | 0.57* | 0.58* |
| Glutamine | 0.84 | 0.91 | 0.65* | 0.8 | 0.81* | 0.68* | 0.71 |
| Palmitoleic acid | 0.2 | 1.28 | 0.66 | 0.55* | 0.37* | 0.71* | 0.39* |
| (C16:cis[9]1) | | | | | | | |
| Campesterol | 0.08 | 0.6* | 0.24* | 0.43* | 0.35* | 0.58* | 0.54* |

TABLE 2
Chemical/physical properties of selected analytes. These biomarkers are characterized
herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 eV): |
| | m/z (%): 204 (100), 73 (18), 205 (16), 206 (7), |
| | 354 (4), 442 (1). |
| 5-O-Methylsphingosine (d18:1) | 5-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 eV): |
| | m/z (%): 250 (100), 73 (34), 251 (19), 354 (14), |
| | 355 (4), 442 (1). |
| Cholesterolester No 01 | Metabolite belongs to the class of cholesterolesters. |
| | It exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 369.2 (+/−0.5). |
| Choline plasmalogen No 01 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 772.6 (+/−0.5). |
| Choline plasmalogen No 02 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 767 (+/−0.5). |
| Choline plasmalogen No 03 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 768.8 (+/−0.5). |
| DAG (C18:1,C18:2) | DAG (C18:1,C18:2) represents the sum parameter of |
| | diacylglycerols containing the combination of a C18:1 |
| | fatty acid unit and a C18:2 fatty acid unit. The mass- |
| | to-charge ratio (m/z) of the ionised species is 641.6 Da |
| | (+/−0.5 Da). |
| Eicosaenoic acid (C20:1) No | Eicosaenoic acid (C20:1) exhibits the following characteristic |
| 02 | ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 eV): |
| | m/z (%): 55 (100), 69 (75), 41 (57), 83 (54), 74 |
| | (53), 97 (45), 110 (20), 292 (13), 293 (13), 124 (12), |
| | 250 (9), 152 (8), 138 (8), 208 (7), 324 (2). |
| Glycerol phosphate, lipid fraction | Glycerol phosphate, lipid fraction represents the sum |
| | parameter of metabolites containing a glycerol-2- |
| | phosphate or a glycerol-3-phosphate moiety and |
| | being present in the lipid fraction after extraction and |
| | separation of the extract into a polar and a lipid fraction. |
| Lysophosphatidylcholine | Lysophosphatidylcholine (C17:0) represents the sum |
| (C17:0) | parameter of lysoglycerophosphorylcholines containing |
| | a C17:0 fatty acid unit. If detected with LC/MS, |
| | applying electro-spray ionization (ESI) mass spectrometry, |
| | the mass-to-charge ratio (m/z) of the positively |
| | charged ionic species is 510.4 Da (+/−0.5 Da). |
| Lysophosphatidylcholine | Lysophosphatidylcholine (C18:0) represents the sum |
| (C18:0) | parameter of lysoglycerophosphorylcholines containing |
| | a C18:0 fatty acid unit. If detected with |
| | LC/MS, applying electro-spray ionization (ESI) mass |
| | spectrometry, the mass-to-charge ratio (m/z) of the |
| | positively charged ionic species is 546.6 Da (+/−0.5 Da). |
| Lysophosphatidylcholine | Lysophosphatidylcholine (C18:1) represents the sum |
| (C18:1) | parameter of lysoglycerophosphorylcholines containing |
| | a C18:1 fatty acid unit. If detected with |
| | LC/MS, applying electro-spray ionization (ESI) mass |
| | spectrometry, the mass-to-charge ratio (m/z) of the |
| | positively charged ionic species is 522.2 Da (+/−0.5 Da). |
| Lysophosphatidylcholine | Lysophosphatidylcholine (C18:2) represents the sum |
| (C18:2) | parameter of lysoglycerophosphorylcholines containing |
| | a C18:2 fatty acid unit. If detected with |
| | LC/MS, applying electro-spray ionization (ESI) mass |
| | spectrometry, the mass-to-charge ratio (m/z) of the |
| | positively charged ionic species is 542.4 Da (+/−0.5 Da). |
| Lysophosphatidylcholine | Lysophosphatidylcholine (C20:4) represents the sum |
| (C20:4) | parameter of lysoglycerophosphorylcholines containing |
| | a C20:4 fatty acid unit. If detected with |
| | LC/MS, applying electro-spray ionization (ESI) mass |
| | spectrometry, the mass-to-charge ratio (m/z) of the |
| | positively charged ionic species is 544.4 Da (+/−0.5 Da). |
| Lysophosphatidylethanolamine | Lysophosphatidylethanolamine (C22:5) exhibits the |
| (C22:5) | following characteristic ionic species when detected |
| | with LC/MS, applying electro-spray ionization (ESI) |
| | mass spectrometry: mass-to-charge ratio (m/z) of the |
| | positively charged ionic species is 528.2 (+/−0.5). |
| Phosphatidylcholine | Phosphatidylcholine (C16:0/C16:0) represents the |
| (C16:0,C16:0) | sum parameter of glycerophosphorylcholines containing |
| | either the combination of of two C16:0 fatty |
| | acid units. The mass-to-charge ratio (m/z) of the ionised |
| | species is 734.8 Da (+/−0.5 Da). |
| Phosphatidylcholine | Phosphatidylcholine (C16:0,C20:5) exhibits the following |
| (C16:0,C20:5) | characteristic ionic species when detected |
| | with LC/MS, applying electro-spray ionization (ESI) |
| | mass spectrometry: mass-to-charge ratio (m/z) of the |
| | positively charged ionic species is 780.8 (+/−0.5). |
| Phosphatidylcholine | Phosphatidylcholine (C16:1,C18:2) represents the |
| (C16:1,C18:2) | sum parameter of glycerophosphorylcholines containing |
| | the combination of a C16:1 fatty acid unit and |
| | a C18:2 fatty acid unit. If detected with LC/MS, applying |
| | electro-spray ionization (ESI) mass spectrometry, |
| | the mass-to-charge ratio (m/z) of the positively |
| | charged ionic species is 756.8 Da (+/−0.5 Da). |
| Phosphatidylcholine | Phosphatidylcholine (C18:0,C18:1) represents the |
| (C18:0,C18:1) | sum parameter of glycerophosphorylcholines containing |
| | the combination of a C18:0 fatty acid unit and |
| | a C18:1 fatty acid unit. If detected with LC/MS, applying |
| | electro-spray ionization (ESI) mass spectrometry, |
| | the mass-to-charge ratio (m/z) of the positively |
| | charged ionic species is 788.6 Da (+/−0.5 Da). |
| Phosphatidylcholine | Phosphatidylcholine (C18:0,C18:2) represents the |
| (C18:0,C18:2) | sum parameter of glycerophosphorylcholines containing |
| | the combination of a C18:0 fatty acid unit and |
| | a C18:2 fatty acid unit. If detected with LC/MS, applying |
| | electro-spray ionization (ESI) mass spectrometry, |
| | the mass-to-charge ratio (m/z) of the positively |
| | charged ionic species is 786.6 Da (+/−0.5 Da). |
| Phosphatidylcholine | Phosphatidylcholine (C18:0,C20:3) exhibits the following |
| (C18:0,C20:3) | characteristic ionic species when detected |
| | with LC/MS, applying electro-spray ionization (ESI) |
| | mass spectrometry: mass-to-charge ratio (m/z) of the |
| | positively charged ionic species is 812.6 (+/−0.5). |
| Phosphatidylcholine | Phosphatidylcholine (C18:0,C20:4) represents the |
| (C18:0,C20:4) | sum parameter of glycerophosphorylcholines containing |
| | the combination of a C18:0 fatty acid unit and |
| | a C20:4 fatty acid unit. If detected with LC/MS, applying |
| | electro-spray ionization (ESI) mass spectrometry, |
| | the mass-to-charge ratio (m/z) of the positively |
| | charged ionic species is 810.8 Da (+/−0.5 Da). |
| Phosphatidylcholine | Phosphatidylcholine (C18:0, C22:6) represents the |
| (C18:0,C22:6) | sum parameter of glycerophosphorylcholines containing |
| | the combination of a C18:0 fatty acid unit and |
| | a C22:6 fatty acid unit. If detected with LC/MS, applying |
| | electro-spray ionization (ESI) mass spectrometry, |
| | the mass-to-charge ratio (m/z) of the positively |
| | charged ionic species is 834.8 Da (+/−0.5 Da). |
| Phosphatidylcholine | Phosphatidylcholine (C16:0/C20:3 C18:1/C18:2) represents |
| (C18:1,C18:2) | the sum parameter of glycerophosphorylcholines |
| | containing the combination of a C18:1 fatty acid |
| | unit and a C18:2 fatty acid unit. The mass-to-charge |
| | ratio (m/z) of the ionised species is 784.6 Da (+/−0.5 Da). |
| Phosphatidylcholine | Phosphatidylcholine (C16:0/C22:6 C18:2/C20:4) represents |
| (C18:2,C20:4) | the sum parameter of glycerophosphorylcholines |
| | containing either the combination of a C16:0 |
| | fatty acid unit and a C22:6 fatty acid unit or the combination |
| | of a C18:2 fatty acid unit and a C20:4 fatty |
| | acid unit. The mass-to-charge ratio (m/z) of the ionised |
| | species is 806.6 Da (+/−0.5 Da). |
| Phosphatidylcholine No 02 | Metabolite belongs to the class of glycerophosphocholines. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 808.4 (+/−0.5). |
| Phosphatidylcholine No 04 | Metabolite belongs to the class of glycerophosphocholines. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 796.8 (+/−0.5). |
| Sphingomyelin (d18:1,C23:0) | Sphingomyelin (d18:1,C23:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 801.8 (+/−0.5). |
| Sphingomyelin (d18:1,C24:0) | Sphingomyelin (d18:1, C24:0) represents the sum |
| | parameter of sphingomyelins containing the combination |
| | of a d18:1 long-chain base unit and a C24:0 |
| | fatty acid unit. If detected with LC/MS, applying electro- |
| | spray ionization (ESI) mass spectrometry, the |
| | mass-to-charge ratio (m/z) of the positively charged |
| | ionic species is 815.8 Da (+/−0.5 Da). |
| Sphingomyelin (d18:2,C16:0) | Sphingomyelin (d18:2,C16:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 723.6 (+/−0.5). |
| Sphingomyelin (d18:2,C18:0) | Sphingomyelin (d18:2,C18:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 729.8 (+/−0.5). |
| TAG (C16:0,C16:1) | Metabolite represents the sum of triacylglycerides |
| | containing the combination of a C16:0 fatty acid unit |
| | and a C16:1 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 549.6 (+/−0.5). |
| TAG (C16:0,C18:1,C18:3) | TAG (C16:0,C18:1,C18:3) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 855.6 (+/−0.5). |
| TAG (C16:0,C18:2) | Metabolite represents the sum of triacylglycerides |
| | containing the combination of a C16:0 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 575.6 (+/−0.5). |
| TAG (C18:1,C18:2) | Metabolite represents the sum of triacylglycerides |
| | containing the combination of a C18:1 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 601.6 (+/−0.5). |
| TAG (C18:2,C18:2) | Metabolite represents the sum of triacylglycerides |
| | containing the combination of a C18:2 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 599.6 (+/−0.5). |
| TAG (C18:2,C18:3) | Metabolite represents the sum of triacylglycerides |
| | containing the combination of a C18:2 fatty acid unit |
| | and a C18:3 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 597.6 (+/−0.5). |
| TAG (DAG-Fragment) | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 600.6 (+/−0.5). |
| TAG No 01 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 547.6 (+/−0.5). |
| TAG No 02 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 695.6 (+/−0.5). |
| TAG No 05 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 879.6 (+/−0.5). |
| TAG No 059 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 904 (+/−0.5). |
| TAG No 07 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 853.6 (+/−0.5). |

TABLE 3

| | Lysmeral | | |
|---|---|---|---|
| Metabolite | m7 | m14 | m28 |

a: Markers for gonadal toxicity (Lysmeral based testes toxicity)
in male rats; Significant up-regulation changes (p-Value ≦0.1)
are marked (*). For some metabolites (marked with #),
additional information are provide in table 2.

| | | | |
|---|---|---|---|
| Lysine | 6.14* | 6.55* | 8.13* |
| Serine | 1.36* | 1.54* | 1.7* |
| Threonine | 1.46* | 1.74* | 2.15* |
| Arginine | 1.37* | 1.59* | 1.76* |

b: Markers for gonadal toxicity (Lysmeral based testes toxicity)
in male rats; Significant down-regulation changes (p-Value ≦0.1)
are marked (*). For some metabolites (marked with #),
additional information are provide in table 2.

| | | | |
|---|---|---|---|
| Lignoceric acid (C24:0) | 0.36 | 0.52* | 0.6* |
| Ceramide (d18:1,C24:0)# | 0.59* | 0.59* | 0.44* |
| 3-O-Methylsphingosine | 0.22 | 0.45* | 0.48* |
| (d18:1)# | | | |
| Sphingomyelin | 0.52* | 0.62* | 0.64* |
| (d18:1,C24:0)# | | | |
| 5-O-Methylsphingosine | 0.19 | 0.48* | 0.48* |
| (d18:1)# | | | |
| Choline plasmalogen No | 0.51* | 0.66* | 0.6* |
| 03# | | | |
| erythro-Sphingosine | 0.21 | 0.51* | 0.51* |
| Palmitic acid (C16:0) | 0.42* | 0.56* | 0.48* |
| threo-Sphingosine | 0.25 | 0.5* | 0.51* |
| Homovanillic acid (HVA) | 0.21* | 0.47* | 0.26* |
| Sphingomyelin | 0.84* | 0.88* | 0.79* |
| (d18:1,C16:0)# | | | |
| alpha-Tocopherol | 0.52* | 0.56* | 0.61* |
| Glutamine | 0.65* | 0.8 | 0.81* |
| Coenzyme Q9 | 0.21* | 0.27* | 0.31* |
| Phosphatidylcholine | 0.74* | 0.79* | 0.83* |
| (C18:1,C18:2)# | | | |
| Myristic acid (C14:0) | 0.54 | 0.59* | 0.53* |
| Sphingomyelin | 0.58* | 0.53* | 0.55* |
| (d18:2,C16:0)# | | | |
| 5-Oxoproline | 0.65* | 0.75* | 0.74* |
| beta-Sitosterol | 0.43* | 0.57* | 0.55* |
| Lysophosphatidylcholine | 0.87* | 0.95* | 0.94* |
| (C20:4)# | | | |
| Dodecanol | 0.45* | 0.75* | 0.82* |
| Phosphatidylcholine | 0.89* | 0.93* | 0.92* |
| (C16:0,C18:2)# | | | |
| Campesterol | 0.24* | 0.43* | 0.35* |
| Oleic acid (C18:cis[9]1) | 0.87 | 1.01 | 0.81* |
| TAG (C16:0,C16:1)# | 0.47 | 0.67* | 0.66* |
| Glutamate | 0.72 | 0.93 | 0.82* |
| 3-Hydroxybutyrate | 0.21* | 0.2* | 0.49* |
| Lysophosphatidylcholine | 0.67* | 0.61* | 0.5* |
| (C20:4)# | | | |

TABLE 4

| | 2- | | | | |
|---|---|---|---|---|---|
| | Methoxyethanol | | Lysmeral | | |
| Metabolite | m7 | m14 | m7 | m14 | m28 |

a: Markers for gonadal toxicity (testes toxicity) in male rats;
Significant changes (p-Value ≦0.05) are marked (*).
For some metabolites (marked with #), additional
information are provide in table 2.

| | | | | | |
|---|---|---|---|---|---|
| Lysine | 1.22* | 1.86 | 6.14* | 6.55* | 8.13* |
| Threonine | 1.72* | 2.93* | 1.46* | 1.74* | 2.15* |
| Histidine | 1.13 | 1.4* | 1.21* | 1.37* | 1.56* |

b: Markers for gonadal toxicity (testes toxicity) in male rats;
Significant changes (p-Value ≦0.05) are marked (*).
For some metabolites (marked with #), additional
information are provide in table 2.

| | | | | | |
|---|---|---|---|---|---|
| Lignoceric acid | 0.61* | 0.46* | 0.36 | 0.52* | 0.6* |
| (C24:0) | | | | | |
| Ceramide | 0.75* | 0.7* | 0.59* | 0.59* | 0.44* |
| (d18:1,C24:0)# | | | | | |
| Sphingomyelin | 0.68* | 0.56* | 0.52* | 0.62* | 0.64* |
| (d18:1,C24:0)# | | | | | |
| erythro-Sphingosine | 0.57* | 0.67* | 0.21 | 0.51* | 0.51* |
| Ceramide | 0.82 | 0.62* | 0.47* | 0.38* | 0.38* |
| (d18:1,C24:1)# | | | | | |
| Sphingomyelin | 0.84* | 0.88* | 0.84* | 0.88* | 0.79* |
| (d18:1,C16:0)# | | | | | |
| Homovanillic acid | NA | NA | 0.21* | 0.47* | 0.26* |
| (HVA) | | | | | |
| 3-Hydroxybutyrate | 0.45* | 0.24* | 0.24* | 0.29* | 0.52* |
| Palmitic acid (C16:0) | 0.92 | 0.75* | 0.42* | 0.56* | 0.48* |
| alpha-Tocopherol | 0.78 | 0.76* | 0.52* | 0.56* | 0.61 |
| Coenzyme Q9 | 0.57* | 0.58* | 0.21* | 0.27* | 0.31* |
| Myristic acid (C14:0) | 0.87 | 0.57* | 0.54 | 0.59* | 0.53* |
| Isopalmitic acid | 1.03 | 0.43* | 0.46* | 0.49* | 0.34* |
| (C16:0) | | | | | |
| Linoleic acid | 1.07 | 0.77* | 0.44* | 0.57* | 0.38* |
| (C18:cis[9,12]2) | | | | | |
| Dodecanol | 0.8 | 0.77* | 0.45 | 0.75* | 0.82 |
| 5-Oxoproline | 0.76* | 0.67* | 0.65* | 0.75 | 0.74* |
| Palmitoleic acid | 0.71* | 0.39* | 0.66 | 0.55* | 0.37* |
| (C16:cis[9]1) | | | | | |
| Campesterol | 0.58* | 0.54 | 0.24* | 0.43* | 0.35* |

TABLE 5a
Markers for gonodal toxicity (testis degeneration) in male rats; Significant up-regulation changes
(p-Value ≦0.2) are marked (*).
For some metabolites (marked with #), additional information are provide in table 2.

| | | | | 2- | | | | | | | | |
|---|---|---|---|---|---|---|---|---|---|---|---|---|
| | 2-Butoxyethanol | | | Methylimidazole | | | Phenylbutazone | | | 2,5-hexanedione | | |
| Metabolite | m7 | m14 | m28 | m7 | m14 | m28 | m7 | m14 | m28 | m7 | m14 | m28 |
| Creatinine | 1.03 | 1.34* | 1.19* | 1.23* | 1.45* | 1.65* | 1.51* | 1.74* | 1.36* | 1.21* | 1.64* | 1.35* |
| Uracil | 0.77* | 0.9 | 0.71* | 0.67* | 0.67* | 0.77* | 0.83* | 0.86 | 0.92 | 0.85* | 0.83* | 0.87* |
| Glucose | 0.81* | 0.73* | 0.93* | 0.86* | 0.88* | 0.87* | 0.89 | 0.92 | 0.83* | 0.85* | 0.89* | 0.85* |
| myo-Inositol | 0.88 | 0.82* | 0.93* | 0.85* | 0.97 | 0.91* | 0.67* | 0.68* | 0.78* | 0.71* | 0.79* | 0.89 |
| Malate | 0.81* | 0.79* | 0.7* | 0.65* | 0.8* | 0.69* | 1.27 | 0.84* | 0.84 | 0.79* | 0.82* | 0.74* |
| Oleic acid | 0.98 | 0.8* | 0.91 | 0.85* | 0.82* | 0.8* | 0.97 | 0.97* | 0.94 | 0.81* | 0.81* | 0.92* |
| (C18:cis[9]1) | | | | | | | | | | | | |
| Spermidine | 0.67* | 0.68* | 0.77* | 0.77* | 0.69* | 0.79* | 0.68* | 0.57* | 0.77* | 0.95 | 0.89* | 0.94 |
| Arachidonic acid | 0.86* | 0.84 | 0.84 | 0.76* | 0.83* | 0.97 | 0.77* | 0.8* | 0.79* | 0.9 | 0.93 | 1.03 |
| (C20:cis[5,8,11,14]4) | | | | | | | | | | | | |
| Alanine | 0.83* | 0.93 | 0.91* | 0.76* | 0.84* | 0.92* | 1.06 | 1.02 | 0.89* | 0.71* | 0.76* | 0.77* |
| Fructose-6- | 0.84 | 1.11 | 0.61* | 0.76 | 1.08 | 0.83* | 0.83* | 1.01 | 1 | 0.75* | 0.7* | 0.69* |
| phosphate | | | | | | | | | | | | |
| DAG (C18:1, C18:2)# | 0.99 | 0.69* | 0.78 | 0.81 | 0.74* | 0.67 | 0.75 | 0.62* | 0.57* | 0.51* | 0.6* | 0.79* |
| Linolenic acid | 1.01 | 0.9* | 0.99 | 0.59* | 0.57* | 0.53* | 0.81* | 0.71* | 0.53* | 0.89* | 0.57* | 0.92* |
| (C18:cis[9,12,15]3) | | | | | | | | | | | | |
| Galactose, lipid fraction | 0.8* | 0.86 | 0.71* | 0.81* | 1 | 1.15 | 0.77* | 0.92 | 1.02 | 0.96 | 0.97 | 0.99 |
| Lactate | 0.93* | 0.86 | 0.72* | 0.68 | 0.69* | 0.77* | 0.86 | 0.7* | 0.85 | 0.82* | 0.69* | 0.66* |
| Phosphate (inorganic | 1.05 | 1.03 | 1.02* | 0.9* | 0.89* | 1.02 | 0.97* | 0.94 | 1.05 | 0.89* | 1 | 1.01 |
| and organic phosphates) | | | | | | | | | | | | |
| Lysine | 0.81* | 1 | 0.86* | 0.81* | 0.97 | 0.88* | 0.97 | 0.92 | 0.88 | 0.96* | 0.93* | 0.96 |
| 18-Hydroxy-11- | 0.18* | 0.44* | 0.68* | 0.49* | 0.59* | 0.58 | 0.25* | 0.18* | 0.4* | 0.89 | 0.33* | 0.24* |
| deoxycorticosterone | | | | | | | | | | | | |
| Heneicosanoic acid | 0.88 | 0.86* | 0.96 | 0.92 | 0.87* | 1.08 | 0.84* | 0.88* | 0.99 | 0.97 | 0.84 | 0.99 |
| (C21:0) | | | | | | | | | | | | |
| TAG (C16:0, C16:1)# | 1.16 | 0.66* | 1.06 | 0.67* | 0.38* | 0.71* | 0.57* | 0.45* | 0.41* | 0.49* | 0.67* | 0.95 |
| Coenzyme Q9 | 0.83* | 0.5* | 0.83 | 0.72* | 0.67* | 0.73* | 0.81 | 0.75* | 0.67* | 0.89 | 1.12* | 1.02 |
| Palmitoleic acid | 0.94 | 0.77 | 0.77 | 0.81 | 0.77* | 0.88* | 0.63* | 0.38* | 0.38* | 0.82* | 1.03 | 1.11 |
| (C16:cis[9]1) | | | | | | | | | | | | |
| Glycerol, polar fraction | 0.86* | 0.95 | 0.87* | 0.75* | 0.84* | 0.79* | 0.98 | 0.95 | 0.93* | 0.96 | 0.98 | 0.8* |
| Glucosamine | 1 | 0.88 | 1.3* | 1 | 1.15 | 0.84 | 0.79 | 1.09 | 0.68* | 0.79* | 0.9 | 0.91* |
| trans-4- | 1.03 | 1.21* | 1.15 | 0.73* | 0.75* | 0.81* | 1.03 | 0.93 | 0.88* | 0.84* | 0.91* | 0.87* |
| Hydroxyproline | | | | | | | | | | | | |
| Phenylalanine | 0.87* | 0.94* | 0.95* | 0.96 | 0.96* | 0.93* | 0.91* | 0.96* | 0.88* | 0.85* | 0.84* | 0.89* |
| Pyruvate | 1.1 | 0.78 | 0.72* | 0.84 | 0.69* | 0.69* | 1.05 | 0.73* | 0.29* | 1.04 | 0.95 | 0.72* |

TABLE 6

| | | | | Raloxifene | | |
|---|---|---|---|---|---|---|
| | Mifepristone | | | Hydrochloride | | |
| Metabolite | f7 | f14 | f28 | f7 | f14 | f28 |

a: Markers for gonodal toxicity (endocrine ovaries toxicity) in
female rats; Significant up-regulation changes (p-Value ≦
0.2) are marked (*). For some metabolites (marked with #),
additional information are provide in table 2.

| | | | | | | |
|---|---|---|---|---|---|---|
| 3- | 1.09 | 1 | 1.09 | 1.15* | 1.37* | 1.09 |
| Methoxytyrosine | | | | | | |

b: Markers for gonodal toxicity (endocrine ovaries toxicity) in
female rats; Significant down regulation changes (p-Value ≦
0.2) are marked (*). For some metabolites (marked with #),
additional information are provide in table 2.

| | | | | | | |
|---|---|---|---|---|---|---|
| Cholesterol | 0.65* | 0.72* | 0.76 | 0.7* | 0.63* | 0.62* |
| Docosapentaenoic acid | 0.67 | 0.97 | 1 | 0.78* | 0.78* | 0.76* |
| (C22:cis[7,10,13,16,19]5) | | | | | | |
| Arachidonic acid | 0.64* | 0.71* | 0.78 | 0.69* | 0.68* | 0.68* |
| (C20:cis[5,8,11,14]4) | | | | | | |
| myo-Inositol, lipid fraction | 0.63* | 0.77* | 0.86 | 0.76* | 0.77* | 0.82* |
| Docosahexaenoic acid | 0.73* | 0.78* | 0.75* | 0.74* | 0.66* | 0.58* |
| (C22:cis[4,7,10,13,16,19]6) | | | | | | |
| threo-Sphingosine | 0.56* | 0.79 | 0.79 | 0.7* | 0.61* | 0.6* |
| gamma-Linolenic acid | 0.76 | 0.69 | 0.73* | 0.62* | 0.77* | 0.6* |
| (C18:cis[6,9,12]3) | | | | | | |
| Phosphatidylcholine | 0.85 | 0.8* | 0.7* | 0.86* | 0.86 | 0.82* |
| (C18:0,C18:1)# | | | | | | |
| Elaidic acid (C18:trans[9]1) | 0.74* | 0.71* | 0.79 | 0.87 | 0.86 | 0.7 |
| Glycerol phosphate, lipid | 0.52* | 0.76 | 0.79 | 0.62* | 0.66* | 0.68* |
| fraction# | | | | | | |
| alpha-Tocopherol | 0.75 | 0.76* | 0.61* | 0.6* | 0.77 | 0.68* |
| Citrate | 0.83* | 0.94 | 0.89* | 0.94 | 0.92* | 0.89* |
| Dodecanol | 0.59* | 0.84* | 0.86 | 1.08 | 0.7* | 0.69* |
| Choline plasmalogen No | 0.89* | 0.77* | 0.74* | 0.78* | 0.83* | 0.75* |
| 03# | | | | | | |
| Lysophosphatidylcholine | 0.93 | 0.93 | 0.87* | 0.91* | 0.9* | 0.86* |
| (C20:4)# | | | | | | |
| Campesterol | 0.61* | 0.66* | 0.83 | 0.46* | 0.36* | 0.28* |
| Phosphatidylcholine No | 0.67* | 0.8* | 0.69* | 0.6* | 0.57* | 0.53* |
| 04# | | | | | | |
| Progesterone | 0.67 | 0.45* | 0.38* | 0.4* | 0.23* | 0.19* |
| Phosphatidylcholine | 0.87* | 0.9 | 0.99 | 0.87* | 0.85* | 0.72* |
| (C18:0,C22:6)# | | | | | | |
| Metanephrine | 0.9 | 0.61 | 0.81 | 1.1 | 0.68* | 0.59* |

TABLE 7
Compounds and dosage administration schemes.

| | | | Dosage ad- | |
|---|---|---|---|---|
| Compound | Synonym | CAS no | ministered | Details |
| 17-alpha- | 17-ethynyl-13-methyl- | 57-63-6 | 200 mg/kg | in corn oil; |
| Ethynyl- | 7,8,9,11,12,13,14,15,16,17- | | by gavage | administration |
| estradiol | decahydro-6H-cyclo- | | | volume: 5 |
| | penta[a]phenanthrene- | | | ml/kg body |
| | 3,17-diol | | | weight |
| 2,5- | 2,5-Diketohexane | 110-13-4 | 0.8 mg/kg | in aqua |
| hexane- | | | body weight | bidest., sub- |
| dione | | | s.c. | cutan; admin- |
| | | | | istration vol- |
| | | | | ume: 1 ml/kg |
| | | | | body weight |
| 2- | na | 111-76-2 | 6,000 ppm | mixture in the |
| Butoxy- | | | in the diet | diet |
| ethanol | | | | |
| 2-Methoxy- | Ethylenglykol- | 109-86-4 | 400 mg/kg | in aqua |
| ethanol | monomethylether | | body weight | bidest., ad- |
| | | | by gavage | ministration |
| | | | | volume: 10 |
| | | | | ml/kg body |
| | | | | weight |
| 2- | na | 693-98-1 | 10,000 ppm | mixture in the |
| Methyl- | | | in the diet | diet |
| imidazole | | | | |
| Lysmeral | 2-Methyl-3(4-tert.- | 80-54-6 | 45 mg/kg | in olive oil; |
| | butylphenyl)-propanal | | body weight | administration |
| | | | by gavage | volume: 5 |
| | | | | ml/kg body |
| | | | | weight |
| Mifepristone | (11b,17b)-11-[4- | 84371-65-3 | 0.3 mg/kg | in drinking |
| | (Dimethylamino)phenyl]- | | body weight | water con- |
| | 17-hydroxy-17-(1-propyn- | | by gavage | taining 1% |
| | yl)estra-4,9-dien-3-one | | | CMC; admin- |
| | | | | istration vol- |
| | | | | ume 10 ml/kg |
| Phenylbutazone | 3,5-dioxo-1,2-diphenyl- | 50-33-9 | 50 mg/kg | in corn oil; |
| | 4-n-butylpyrazolidine | | body weight | administration |
| | | | by gavage | volume: 5 |
| | | | | ml/kg body |
| | | | | weight |
| Raloxifene | Evista | 82640-04-8 | 600 mg/kg | in drinking |
| Hydrochloride | | | body weight | water con- |
| | | | by gavage | taining 0.5% |
| | | | | CMC (Tylose |
| | | | | CB30000), |
| | | | | administration |
| | | | | volume: 10 |
| | | | | ml/kg bw |

1. A method for diagnosing gonadal toxicity comprising:
(a) determining the amount of at least one biomarker selected from any one of Tables 1a, 1b, 3a, 3b, 4a, 4b, 5a, 5b, 6a or 6b in a test sample of a subject suspected to suffer from gonadal toxicity, and
(b) comparing the amounts determined in step (a) to a reference, whereby gonadal toxicity is to be diagnosed.

2. The method of claim 1, wherein said subject has been brought into contact with a compound suspected to be capable of inducing gonadal toxicity.

3. A method of determining whether a compound is capable of inducing gonadal toxicity in a subject comprising:
(a) determining in a sample of a subject which has been brought into contact with a compound suspected to be capable of inducing gonadal toxicity the amount of at least one biomarker selected from any one of Tables 1a, 1b, 3a, 3b, 4a, 4b, 5a, 5b, 6a or 6b; and
(b) comparing the amounts determined in step (a) to a reference, whereby the capability of the compound to induce gonadal toxicity is determined.

4. The method of claim 2, wherein said compound is at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

5. The method of claim 1, wherein said reference is derived from (i) a subject or group of subjects which suffers from gonadal toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

6. The method of claim 5, wherein essentially identical amounts for the biomarkers in the test sample and the reference are indicative for gonadal toxicity.

7. The method of claim 1, wherein said reference is derived from (i) a subject or group of subjects known to not suffer from gonadal toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

8. The method of claim 1, wherein said reference is a calculated reference for the biomarkers for a population of subjects.

9. The method of claim 7, wherein amounts for the biomarkers which differ in the test sample in comparison to the reference are indicative for gonadal toxicity.

10. A method of identifying a substance for treating gonadal toxicity comprising the steps of:
(a) determining in a sample of a subject suffering from gonadal toxicity which has been brought into contact with a candidate substance suspected to be capable of treating gonadal toxicity the amount of at least one biomarker selected from any one of Tables 1a, 1b, 3a, 3b, 4a, 4b, 5a, 5b, 6a or 6b; and
(b) comparing the amounts determined in step (a) to a reference, whereby a substance capable of treating gonadal toxicity is to be identified.

11. The method of claim 10, wherein said reference is derived from (i) a subject or group of subjects which suffers from gonadal toxicity or (ii) a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

12. The method of claim 11, wherein amounts for the biomarkers which differ in the test sample and the reference are indicative for a substance capable of treating gonadal toxicity.

13. The method of claim 10, wherein said reference is derived from (i) a subject or group of subjects known to not suffer from gonadal toxicity or (ii) a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

14. The method of claim 10, wherein said reference is a calculated reference for the biomarkers in a population of subjects.

15. The method of claim 13 or 14, wherein essentially identical amounts for the biomarkers in the test sample and the reference are indicative for a substance capable of treating gonadal toxicity.

16. (canceled)

17. A device for diagnosing gonadal toxicity in a sample of a subject suspected to suffer therefrom comprising:
(a) an analyzing unit comprising a detection agent for at least one biomarker selected from any one of Tables 1a, 1b, 3a, 3b, 4a, 4b, 5a, 5b, 6a or 6b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby gonadal toxicity is diagnosed.

18. The device of claim 17, wherein said stored reference is a reference derived from a subject or a group of subjects known to suffer from gonadal toxicity or a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an essentially identical amount of the at least one biomarker in the test sample in comparison to the reference is indicative for the presence of gonadal toxicity or wherein an amount of the at least one biomarker in the test sample which differs in comparison to the reference is indicative for the absence of gonadal toxicity.

19. The device of claim 17, wherein said stored reference is a reference derived from a subject or a group of subjects known to not suffer from gonadal toxicity or a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride and said data processor executes instructions for comparing the amount of the at least one biomarker determined by the analyzing unit to the stored reference, wherein an amount of the at least one biomarker in the test sample which differs in comparison to the reference is indicative for the presence of gonadal toxicity or wherein an essentially identical amount of the at least one biomarker in the test sample in comparison to the reference is indicative for the absence of gonadal toxicity.

20. A kit for diagnosing gonadal toxicity comprising a detection agent for at least one biomarker selected from any of Tables 1a, 1b, 3a, 3b, 4a, 4b, 5a, 5b, 6a or 6b and standards for the said at least one biomarker the concentration of which is derived from (i) a subject or a group of subjects known to suffer from goodal toxicity or a subject or group of subjects which has been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride or derived (ii) from a subject or a group of subjects known to not suffer from gonadal toxicity or a subject or group of subjects which has not been brought into contact with at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.

21. The method of claim 3, wherein said compound is at least one compound selected from the group consisting of: 17-alpha-Ethynylestradiol, Lysmeral, 2-Methoxyethanol, 2-Butoxyethanol, 2-Methylimidazole, Phenylbutazone, 2,5-Hexanedione, Mifepristone and Raloxifene Hydrochloride.
